(19) United States
(12) Patent Application Publication
(10) Pub. No.: US 2023/0025475 A1
(43) Pub. Date: Jan. 26, 2023

(54) SYSTEMS AND METHODS FOR REAL-TIME SAMPLING

(71) Applicant: OLYMPUS MEDICAL SYSTEMS CORPORATION, HACHIOJI-SHI (JP)

(72) Inventors: Madeline C. Graham, Sammamish, WA (US); Jason T. Panzenbeck, Redmond, WA (US)

(21) Appl. No.: 17/813,227

(22) Filed: Jul. 18, 2022

Related U.S. Application Data
(60) Provisional application No. 63/223,743, filed on Jul. 20, 2021.

(51) Int. Cl. A61B 10/04; A61B 8/12; A61B 1/01; A61B 1/267; A61B 8/00; A61B 1/018; A61B 1/005; A61B 1/00

(52) U.S. Cl. CPC A61B 10/04; A61B 1/01; A61B 1/018; A61B 1/0052; A61B 1/00148; A61B 1/2676; A61B 8/12; A61B 8/56; A61B 8/445; A61B 8/461; A61B 2010/045

(57) ABSTRACT

Apparatuses, systems, and methods for controlling the sampling of tissue using a guidewire. In an illustrative embodiment, an apparatus includes a handle, a flexible dual lumen catheter couplable to the handle, and a guidewire guide disposed at a distal end of the flexible dual lumen catheter.

### CROSS-REFERENCE TO RELATED APPLICATION

This application claims the benefit of priority to U.S. Provisional Pat. Application Serial No. 63/223,743, filed Jul. 20, 2021, the contents of which are incorporated herein by reference in their entirety.

### DESCRIPTION OF THE RELATED ART

The medical devices that are currently available for ultrasound visualization and sampling of peripheral lung tumors are limited by how deep they can penetrate into the lung. Typically, during peripheral sampling a real-time sampling device is fed through a bronchoscope of a particular size for receiving the real-time sampling device. The bronchoscope used for delivery of the real-time sampling device is too big to go much further than secondary airway branches within the lung.

The statements in this section merely provide background information related to the present disclosure and may not constitute prior art.

### BRIEF SUMMARY OF EMBODIMENTS

Disclosed embodiments include apparatuses, systems, and methods for controlling the sampling of tissue using a guidewire. The inventors have discovered, among other things, that use of a guide wire and sampling system with a guide wire guide can be used to obtain tissue samples from much deeper into the lungs. In addition to the embodiments discussed in detail herein, the invention can be used within the working channel of devices, such as a robotic catheter due to the reduced size requirements through use of a guide wire and guide wire guide.

In an illustrative embodiment, an apparatus includes a handle, a flexible dual lumen catheter couplable to the handle, and a guidewire guide disposed at a distal end of the flexible dual lumen catheter.

In another illustrative embodiment, a system includes an imaging system, a guidewire system, and a real-time sampling system. The imaging system includes a radial ultrasound probe. The guidewire system includes a guidewire and a scope having a single working channel. The real-time sampling system includes a handle, a flexible dual lumen catheter couplable to the handle, and a guidewire guide disposed at a distal end of the flexible dual lumen catheter, wherein the guidewire guide is configured to slidably receive the guidewire.

In still another illustrative embodiment, a method includes providing a dual lumen catheter and providing a guidewire guide disposed at least near a distal end of the dual lumen catheter.

In one embodiment, a method visually detects target tissue, such as a lesion, a tumor, or the like, using an imaging device deployed from a single working channel endoscope. Once the target tissue has been detected, the imaging device is removed, and a guidewire is delivered through the single working channel endoscope and anchored adjacent the target tissue. The single working channel endoscope or an ultra-thin bronchoscope has a smaller outer diameter than a traditional multi-lumen endoscope or bronchoscope, thus allowing for travel to deeper and smaller air passageways. The single working channel endoscope is then removed leaving only the guidewire. A guidewire guide located on a dual-lumen or real-time sampling catheter is passed over a proximal end of the anchored guidewire, then advanced until adjacent the target tissue. A radial ultrasound probe is inserted into a lumen of the dual lumen catheter, then the target tissue is reacquired with the radial ultrasound probe. A tissue sampling tool is advanced through the other lumen of the dual lumen catheter, out a distal port of the dual lumen catheter, and into the target tissue. Then, the tissue sampling tool and the sampled tissue are removed for analysis.

Further features, advantages, and areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader’s understanding of the disclosed technology and shall not be considered limiting of the breadth, scope, or applicability thereof. It should be noted that for clarity and ease of illustration these drawings are not necessarily made to scale.

FIG. 1 is a perspective view and block diagram of an illustrative bronchoscope system.

FIG. 2 is a side view and block diagram of a real-time sampling system;

FIG. 3 is a perspective view of a portion of the bronchoscope with imaging components within an airway performing a first function.

FIG. 4 is a side view of an illustrative guidewire.

FIG. 5 is a perspective view of a portion of the bronchoscope and the guidewire of FIG. 4 within the airway performing a second function.

FIG. 6 is a side, x-ray view of a distal end of the real-time sampling system of FIG. 2.

FIGS. 7 and 8 are side views of the real-time sampling system of FIG. 2 in a tissue sampling operation.

FIG. 9 is a flow diagram of an illustrative method for performing tissue sampling.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

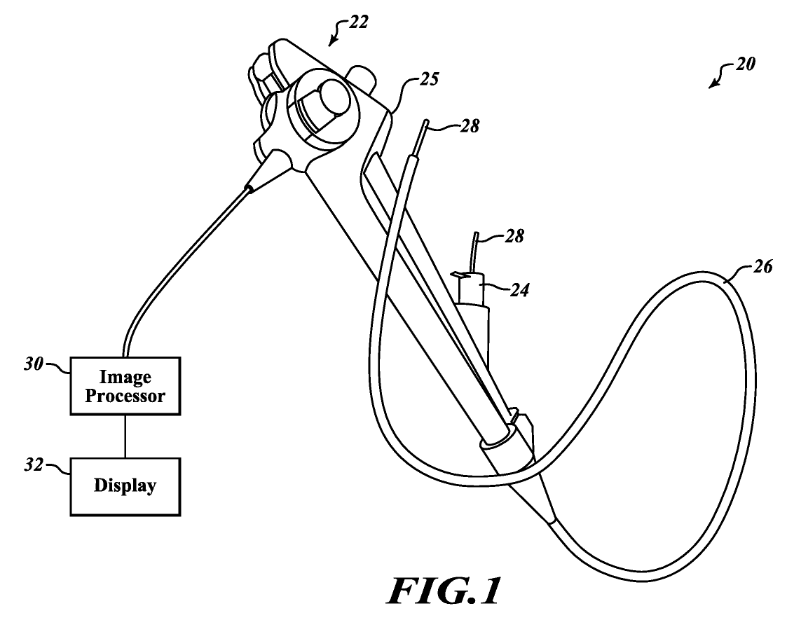

Referring now to FIG. 1, a bronchoscope system 20 includes a bronchoscope 22 with an insertion tube 26, and image processor 30, and a display device 32. The bronchoscope 22 includes a camera that provides image data to the image processor 30 for generation of an image for presentation on the display device 32. A radial ultrasound probe 28 is slidably received within the bronchoscope 22 via a port 24 of a handle 25. The radial ultrasound probe 28 also maybe in data communication with the image processor 30.

The display device 32 is in wired or wireless signal communication with the image processor 30. The display device 32 presents images based on information received from the camera of the bronchoscope 22 and/or a radial ultrasound transducer at the distal end of the radial ultrasound probe 28. A diagnostic bronchoscope (e.g., BF-MP190F produced by Olympus®) is an example of the bronchoscope 22 and the radial endobronchial ultrasound (EBUS) mini-probes produced by Olympus® are examples of the radial ultrasound probes 28. The bronchoscope 22 may include a 3.0 mm distal-end outer diameter with a 1.7 mm working channel, thereby facilitating deep access into the lung. Other types of endoscopes may be used such as an endoscope with an outer diameter less than 4.0 mm, less than 3.5 mm, less than 3.2 mm, less than 3.0 mm, and the like.

A guidewire (not shown) may be received within the handle 25 and the insertion tube 26 via the port 24. The guidewire will be described in more detail below.

In another example, the EBUS could be adapted or replaced by a piezoelectric micromachined ultrasonic transducer (PMUT) or a capacitive micromachined ultrasonic transducer (CMUT). The PMUT or CMUT based sensor allows for further miniaturization of the imaging device and enables use through even smaller catheter systems. In an example, the PMUT or CMUT based sensor could be used via a robotic catheter system to locate target tissue. A guide wire could then be placed as discussed below, and a dual lumen catheter then guided through a working channel of the robotic catheter over the small guide wire to the target tissue for sampling.

In various embodiments, the insertion tube 26 of the bronchoscope 22 is inserted into the location of a suspected tumor or lesion. Then, the radial ultrasound probe 28 is passed through the bronchoscope and out the distal end. The radial ultrasound probe 28 provides image data for processing by the image processor 30 and display on the display device 32. Once the displayed images show the presence of any target tissue, i.e., lesions, tumors, the radial ultrasound probe 28 is removed from the bronchoscope 22. While keeping the insertion tube 26 in place, the guidewire is passed through the working channel of the bronchoscope 22 to a distance within the lung slightly distal of where the target tissue was identified. The guidewire is anchored within the lung, then the bronchoscope 22 is removed leaving the guidewire within the lung.

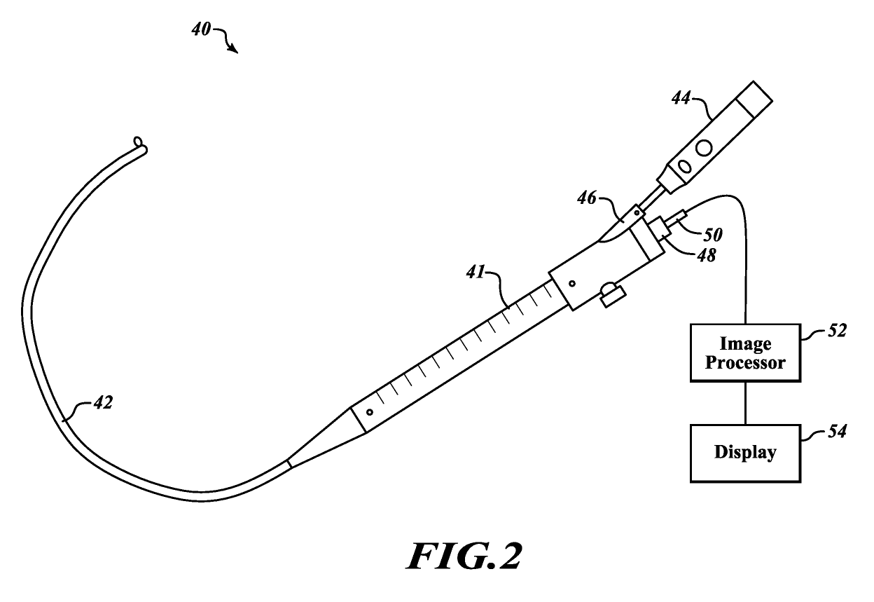

Referring to FIG. 2, a multi-lumen catheter device 40 includes a handle 41 and a flexible insertion tube 42. The multi-lumen catheter device 40 may receive an imaging device 50, such as without limitation, a radial ultrasound probe, that is receivable within a first lumen of the multi-lumen catheter device 40. In another example, the imaging device 50 is a PMUT or a CMUT based ultrasound probe. The PMUT or CMUT based sensors can operate in a manner similar to the following discussion related to the radial ultrasound probe. Although, PMUT and/or CMUT based sensors are typically not designed to generate a 360-degree image as may be provided by the radial ultrasound probe. In the present application, a directional ultrasound probe, such as a PMUT or CMUT, is useful in providing guidance to the sampling needle (which is dependent on properly radial orientation). The imaging device 50 includes a radial ultrasound transducer that is in data communication with an image processor 52. The image processor 52 is in data communication with a display device 54 for processing data received. The display device 54 presents images in response to receiving the processed image data from the image processor 52. A medical device 44 having a handle and a flexible active portion is receivable within a port 46 of the handle 41 which leads to a second lumen of the flexible insertion tube 42. The distal end of the flexible insertion tube 42 includes an exit port for allowing the medical device 44 to exit the second lumen to procure a tissue sample at a desired location within a body. The medical device 44 also may include a stylet (not shown in FIG. 2) that may be removably insertable into and/or through the needle. The medical device 44 may include biopsy forceps, cytology brushes, needles, or the like.

In various embodiments, the flexible insertion tube 42 is fixedly secured to the handle 41 while the medical device 44 and the imaging device 50 are slidably received into the flexible insertion tube 42 via the handle 41.

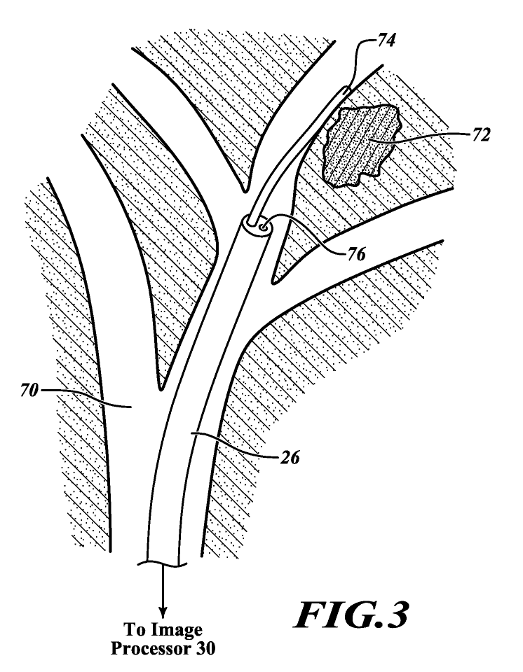

Referring to FIG. 3, the insertion tube 26 of the bronchoscope 22 is inserted into an airway 70 to a suspected location within the lung where target tissue 72 may be located or as deep into the lung as the insertion tube 26 will go. A distal end 74 of the radial ultrasound probe 28 is passed through a working channel of the bronchoscope 22 and out a distal end of the insertion tube 26. The distal end of the insertion tube 26 may include a camera 76 and illumination devices/lights and/or fiber optic wires. The camera 76 delivers the image data to the display 32 via the image processor 30 provide live feedback to an operator.

In the robotic catheter example, the robotically steerable catheter and fiber optic imaging can be used to assist in the positioning of the distal end 74 of the ultrasound probe 28 near the target tissue 72. In this example, the insertion tube 26 would be the working channel of a robotic catheter device.

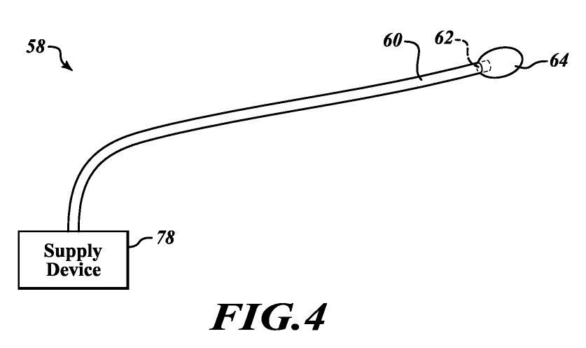

Referring to FIG. 4, a guidewire 58 includes an internal lumen 60 that extends from a proximal end to a distal port 62. Surrounding the distal end of the guidewire 58 is an anchoring mechanism, such as, without limitation, an inflatable balloon 64. The proximal end of the guidewire 58 is configured to attach to a gas or fluid supply device 78. The gas or fluid supply device 78 may be a syringe, an air pump, or the like. Wires/tubes having a lumen with a balloon are well known in the art and no further explanation is necessary for a person of skill in the art to understand the disclosed subject matter.

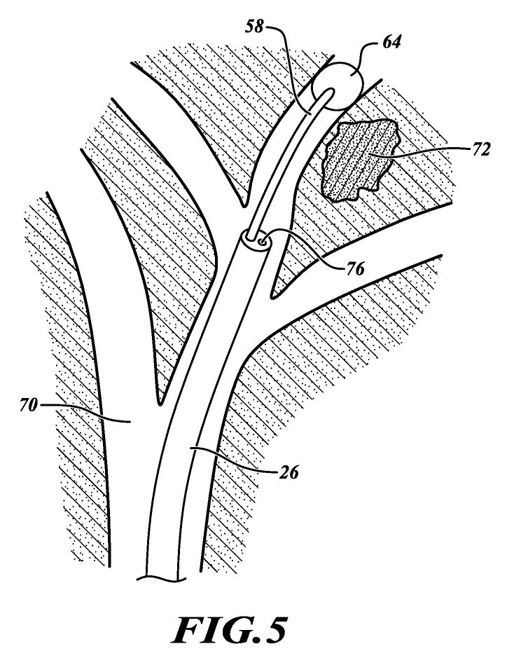

Referring to FIG. 5, after the radial ultrasound probe 28 has identified location of the target tissue 72 using the bronchoscope 22, the radial ultrasound probe 28 is removed from the working channel of the bronchoscope 22. Then, the guidewire 58 is inserted into the working channel of the bronchoscope 22 while maintaining the insertion tube 26 in the same position it was during use of the radial ultrasound probe 28. The guidewire 58 is extended beyond the distal end of the insertion tube 26 past a location where the target tissue 72 was identified. The balloon 64 is inflated using the supply device 78. The inflated balloon 64 applies pressure to the walls of the airway 70, thus anchoring the guidewire 58 in place.

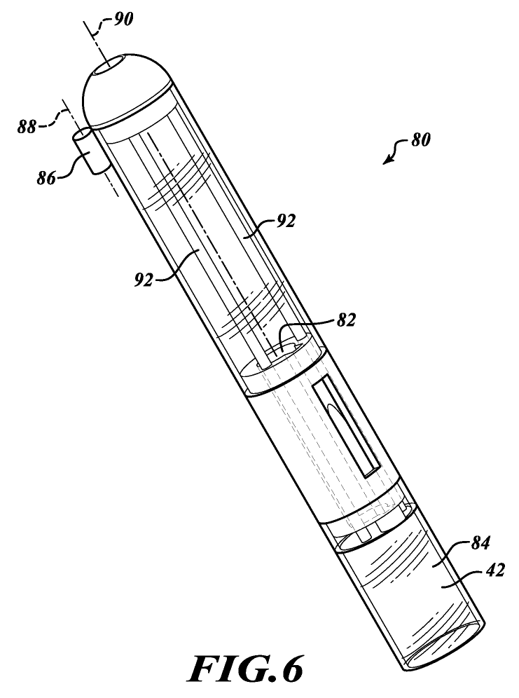

Referring additionally to FIG. 6, a distal end 80 of the flexible insertion tube 42 of the dual-lumen catheter 40 includes a first lumen 82 configured to receive the radial ultrasound probe 50 and a second lumen 84 configured to receive the medical device 44. The second lumen 84 includes a ramp and an exit port at a distal end. The ramp is configured to deflect the medical device 44 as it exits the exit port. The proximity of the exit port to the first lumen 82 allows the ultrasound probe 50 to provide an ultrasonic image of the medical device 44 as it exits the exit port. This potentially allows an operator to see the medical device 44 as it enters into target tissue. In various embodiments, the distal end 80 includes orientation pins 92 located on the same half of the distal end 80 as the ramp and exit port. Images produced by the radial ultrasound probe 50 received within the first lumen 82 produce images with feedback from the orientation pins 92 and the medical device 44. The feedback of the medical device 44 would appear within a short angle between the feedback of the orientation pins 92. In this example, the orientation pins 92 provide guidance as to the position and orientation of the distal end 80 to ensure an understanding of where the medical device 44 will exit the exit port thereby assisting in obtaining a sample of the target tissue.

Attached at or near the distal end 80 of the insertion tube 42 is a guidewire tracking device 86. The guidewire tracking device 86 may be located on a side of the distal end 80 opposite from the exit port. This keeps the guidewire tracking device 86 from interfering with ultrasound images of the orientation pins 92 and the medical device 44. In various embodiments, the guidewire tracking device 86 may be a closed or open (helical) guide or loop device configured to slidably receive the guidewire 58. The guidewire tracking device 86 may include a longitudinal axis that is approximately parallel to a longitudinal axis of the distal end of the insertion tube 42. The guidewire tracking device 86 may be formed of a medical grade material having a compliant or non-compliant form. The guidewire tracking device 86 may be frictionally attached with a heat shrink material, attached with a medical grade adhesive, molded simultaneously with the forming of the insertion tube 42, or the like. The guidewire tracking device 86 may include a single loop or tube formed from a metal or plastic wire.

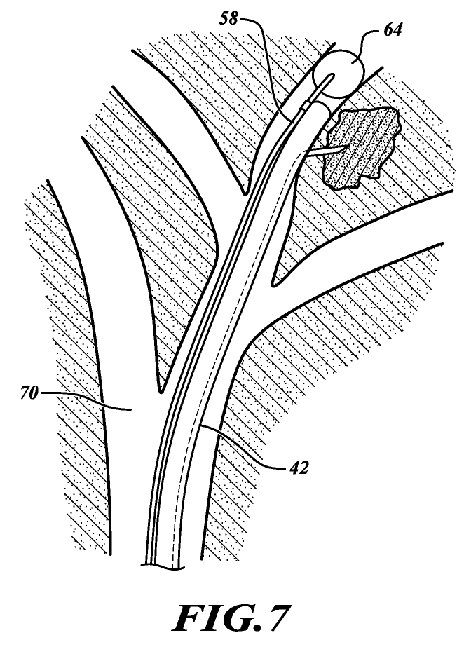

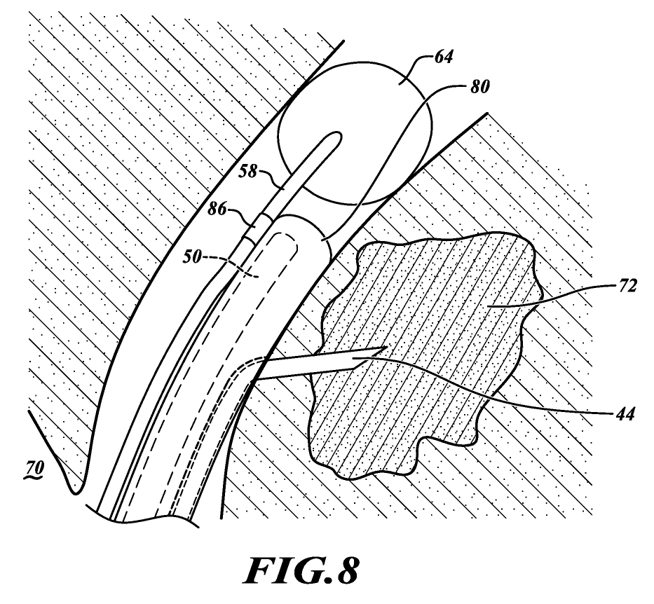

Referring additionally to FIGS. 7 and 8, the bronchoscope 22 has been removed while the guidewire 58 remains anchored by the balloon 64 within the airway 70. The insertion tube 42 of the real-time sampling device 40 has been advanced to a location near the target tissue 72 by virtue of the guidewire tracking device 86 receiving the guidewire 58. Fluoroscopy may be used to allow an operator to visually track the insertion tube 42 relative to the guidewire 58. Once the distal end of the insertion tube 42 is at or near the location of the target tissue 72, the radial ultrasound probe 50 is advanced to the distal end of the first lumen 82. Once the image presented on the display device 54 from the radial ultrasound probe 50 visualizes the target tissue 72, the medical device 44 is passed through the second lumen 84, out the exit port, and into the imaged target tissue 72. The medical tool 44 may be repeatedly inserted into the target tissue 72. Once the operator has completed sampling of the target tissue 72, all the devices within the airway 70 are removed.

When utilizing ultrasonic imaging, it is necessary to transmit the ultrasound waves through a proper medium, typically a fluid or gel in contact with the tissue to be imaged. Alternatively, the ultrasound sensor is placed in direct contact with the tissue to be imaged. As the present device is designed for use within an airway of a patient, no fluid is likely to be present. Accordingly, the ultrasound imaging sensor will need to be placed in contact with a sidewall of the airway adjacent the target tissue. In this example, the anchor mechanism (balloon) assists in ensuring that the ultrasound probe (e.g., imaging device 50) within the distal end 80 is positioned against the tissue wall to ensure proper ultrasound transmission. As illustrated, one method of assisting in positioning the distal end 80 is to offset the guide wire connection with the balloon to bias the position within the airway (lumen). The position of the guide wire guide on the distal end 80 also plays a key role in biasing the imaging device 50 into a proper position against the tissue side wall. FIG. 8 illustrates an example of the imaging device 50 within the distal end 80 biased against the sidewall of an airway adjacent to the target tissue 72. Note, the portion of the distal end 80 containing the imaging device 50 is a suitable medium for passing ultrasonic waves for imaging purposes.

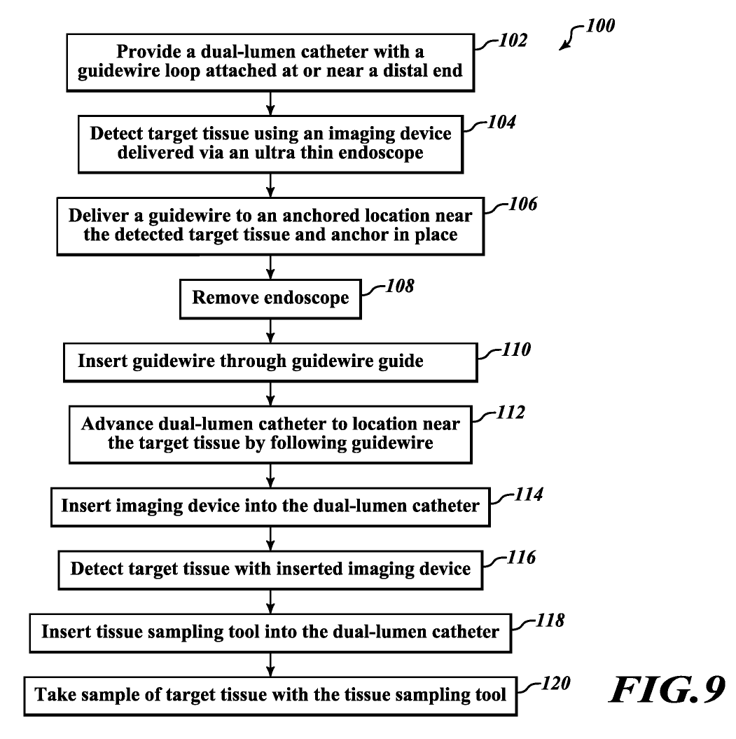

Referring to FIG. 9, a process 100 for performing tissue sampling is provided. At a block 102, a dual-lumen catheter with a guidewire guide attached at or near a distal end is provided. At a block 104, target tissue is detected using an imaging device delivered via an ultra-thin endoscope. At a block 106, a guidewire is delivered to a location near the detected target tissue and anchored in place. At a block 108, the endoscope is removed leaving the guidewire in place. At a block 110, the guidewire is inserted through a guidewire guide on a dual-lumen catheter. At a block 112, the dual-lumen catheter is advanced to location near the target tissue by following the guidewire. At a block 114, an imaging device is inserted into the dual-lumen catheter. At a block 116, target tissue is detected with the inserted imaging device. At a block 118, a tissue sampling tool is inserted into the dual-lumen catheter. At a block 120, a sample of the target tissue is taken with the tissue sampling tool, then all the inserted components are removed.

### Embodiments

A. An apparatus comprising: a handle; a flexible dual lumen catheter couplable to the handle; and a guidewire guide disposed at a distal end of the flexible dual lumen catheter.

B. The apparatus of A, wherein the flexible dual lumen catheter includes: a first lumen configured to slidably receive an imaging device; and a second lumen configured to slidably receive a medical tool.

C. The apparatus of B, wherein the handle includes: a first port and a first lumen configured to receive the imaging device; and a second port and a second lumen configured to receive the medical tool.

D. The apparatus C, wherein the second lumens and the second port are configured to slidably receive a sampling needle.

E. The apparatus of C, wherein the first lumens and the first port are configured to slidably receive a radial ultrasound probe.

F. The apparatus of A-E, wherein: the guidewire guide includes a first longitudinal axis; a portion of the flexible dual lumen catheter adj acent to an attachment point with the guidewire guide includes a second longitudinal axis, and the first longitudinal axis and the second longitudinal axis are parallel.

G. A system comprising: an imaging system including: a radial ultrasound probe; a guidewire system including: a guidewire; and a scope comprising a single working channel; and a real-time sampling system including: a handle; a flexible dual lumen catheter couplable to the handle; and a guidewire guide disposed at a distal end of the flexible dual lumen catheter, wherein the guidewire guide is configured to slidably receive the guidewire.

H. The system of G, wherein: the real-time sampling system further includes a medical tool; and the flexible dual lumen catheter includes: a first lumen configured to slidably receive the radial ultrasound probe; and a second lumen configured to slidably receive the medical tool.

I. The system H, wherein the medical tool includes a sampling needle.

J. The system of H-I, wherein the handle includes: a first port and a first lumen configured to slidably receive the radial ultrasound probe; and a second port and a second lumen configured to slidably receive the medical tool.

K. The system of G-J, wherein the imaging system further includes: an image processor configured to: receive image data from the radial ultrasound probe; and generate images responsive to the received image data; and a display device being in data communication with the image processor being configured to generate a display responsive to the generated images.

L. The system of G-K, wherein the scope includes: a handle including a port and a lumen configured to separately receive the radial ultrasound probe and the guidewire; and a flexible catheter couplable to the handle, the flexible catheter including a lumen and an outer diameter less than 3.2 mm.

M. The system of Claim 7, wherein: the guidewire guide includes a first longitudinal axis; and a portion of the flexible dual lumen catheter adjacent to an attachment point with the guidewire guide includes a second longitudinal axis, the first longitudinal axis and the second longitudinal axis are parallel.

N. A method comprising: providing a dual lumen catheter; and providing a guidewire guide disposed at least near a distal end of the dual lumen catheter.

O. The method of N, wherein providing the guidewire guide includes: attaching the guidewire guide at least near the distal end of the dual lumen catheter, such that a first longitudinal axis of the guidewire guide is parallel to a second longitudinal axis of the dual lumen catheter.

P. The method of N-O, further comprising: visually detecting target tissue using an imaging device deployed from a single working channel endoscope.

Q. The method of P, further comprising: delivering a guidewire to a location within an airway near the detected target tissue via the single working channel endoscope, wherein the single working channel endoscope includes an insertion tube with an outer diameter less than 3.2 mm; anchoring the guidewire within the air passageway; removing the endoscope; passing the guidewire guide over a proximal end of the anchored guidewire; and advancing the dual lumen catheter within the airway adjacent to the detected target tissue responsive to the guidewire guide traversing over the anchored guidewire.

R. The method of Q, further comprising: inserting a radial ultrasound probe within a lumen of the dual lumen catheter; imaging the target tissue with the radial ultrasound probe.

S. The method of R, further comprising: advancing a tissue sampling tool through the other lumen of the dual lumen catheter, out a distal port of the dual lumen catheter, and into the imaged target tissue.

T. The method of Q, wherein anchoring includes inflating a balloon at a distal end of the guidewire.

While various embodiments of the disclosed technology have been described above, it should be understood that they have been presented by way of example only, and not of limitation. Likewise, the various diagrams may depict an example construction or other configuration for the disclosed technology, which is done to aid in understanding the features and functionality that can be included in the disclosed technology. The disclosed technology is not restricted to the illustrated example construction or configurations, but the desired features can be implemented using a variety of alternative construction and configurations. Indeed, it will be apparent to one of skill in the art how alternative functional, logical, or physical partitioning and configurations can be implemented to implement the desired features of the technology disclosed herein. Also, a multitude of different constituent parts names other than those depicted herein can be applied to the various parts. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or "an" should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

What is claimed is:

1. A system comprising:
an imaging system including:
a radial ultrasound probe;
a guidewire system including:
a guidewire; and
a scope comprising a single working channel; and
a real-time sampling system including:
a handle;
a flexible dual lumen catheter couplable to the handle; and
a guidewire guide disposed at a distal end of the flexible dual lumen catheter, wherein the guidewire guide is configured to slidably receive the guidewire.

2. The system of claim 1, wherein:
the real-time sampling system further includes a medical tool; and
the flexible dual lumen catheter includes:
a first lumen configured to slidably receive the radial ultrasound probe; and
a second lumen configured to slidably receive the medical tool.

3. The system claim 2, wherein the medical tool includes a sampling needle.

4. The system of claim 2, wherein the handle includes:
a first port and a first lumen configured to slidably receive the imaging device; and
a second port and a second lumen configured to slidably receive the medical tool.

5. The system of claim 1, wherein the imaging system further includes:
an image processor configured to:
receive image data from the radial ultrasound probe; and
generate images responsive to the received image data; and
a display device being in data communication with the image processor being configured to generate a display responsive to the generated images.

6. The system of claim 1, wherein the scope includes:
a handle including a port and a lumen configured to separately receive the radial ultrasound probe and the guidewire; and
a flexible catheter couplable to the scope handle, the flexible catheter including a lumen and an outer diameter less than 3.2 mm.

7. The system of claim 1, wherein:
the guidewire guide includes a first longitudinal axis; and
a portion of the flexible dual lumen catheter adjacent to an attachment point with the guidewire guide includes a second longitudinal axis,
the first longitudinal axis and the second longitudinal axis are parallel.

8. The system of claim 1, wherein the guide wire includes an anchoring mechanism disposed on a distal end, the anchoring mechanism configured to secure the guide wire within an airway or similar lumen within a patient.

9. The system of claim 8, wherein the guide wire is coupled to the anchor mechanism in an offset manner to bias a distal end of the flexible dual lumen catheter against a tissue sidewall adjacent the target tissue.

10. The system of claim 9, wherein a portion the distal end of the flexible dual lumen catheter distal of an exit port for the second lumen is ultrasonically transmissible.

11. An apparatus comprising:
a handle;
a flexible dual lumen catheter couplable to the handle; and
a guidewire guide disposed at a distal end of the flexible dual lumen catheter.

12. The apparatus of claim 11, wherein the flexible dual lumen catheter includes:
a first lumen configured to slidably receive an imaging device; and
a second lumen configured to slidably receive a medical tool.

13. The apparatus of claim 12, wherein the handle includes:
a first port and a first lumen configured to receive the imaging device; and
a second port and a second lumen configured to receive the medical tool.

14. The apparatus claim 13, wherein the second lumen and the second port are configured to slidably receive a sampling needle.

15. The apparatus of claim 13, wherein the first lumen and the first port are configured to slidably receive a radial ultrasound probe.

16. The apparatus of claim 11, wherein:
the guidewire guide includes a first longitudinal axis;
a portion of the flexible dual lumen catheter adjacent to an attachment point with the guidewire guide includes a second longitudinal axis, and
the first longitudinal axis and the second longitudinal axis are parallel.

17. A method comprising:
providing a dual lumen catheter;
attaching a guidewire guide at least near the distal end of the dual lumen catheter, such that a first longitudinal axis of the guidewire guide is parallel to a second longitudinal axis of the dual lumen catheter;
delivering a guidewire to a location within an airway near a detected target tissue via a single working channel endoscope, wherein the single working channel endoscope includes an insertion tube with an outer diameter less than 3.2 mm;
anchoring the guidewire within the air passageway;
removing the endoscope;
passing the guidewire guide over a proximal end of the anchored guidewire; and
advancing the dual lumen catheter within the airway adjacent to the detected target tissue responsive to the guidewire guide traversing over the anchored guidewire.

18. The method of claim 17, further comprising:
inserting a radial ultrasound probe within a lumen of the dual lumen catheter;
imaging the target tissue with the radial ultrasound probe.

19. The method of claim 18, further comprising:
advancing a tissue sampling tool through the other lumen of the dual lumen catheter, out a distal port of the dual lumen catheter, and into the imaged target tissue.

20. The method of claim 17, wherein anchoring includes inflating a balloon at a distal end of the guidewire.
